(19) United States
(12) Patent Application Publication
(10) Pub. No.: US 2017/0073304 A1
(43) Pub. Date: Mar. 16, 2017

(54) METHOD FOR THE PREPARATION OF TERIFLUNOMIDE

(71) Applicant: Formosa Laboratories Inc., Taoyuan (TW)

(72) Inventors: CHING-PENG WEI, Taoyuan (TW); PEI-CHEN HUANG, Taoyuan (TW)

(73) Assignee: FORMOSA LABORATORIES INC., Taoyuan (TW)

(21) Appl. No.: 14/849,808

(22) Filed: Sep. 10, 2015

(51) Int. Cl. C07C 253/00

(52) U.S. Cl. CPC C07C 253/00

(57) ABSTRACT

The present invention relates to a method for preparation of Teriflunomide, comprising steps of: (a) adding Leflunomide to an alcoholic solvent to give solution (I); (b) adding an aqueous sodium hydroxide solution slowly into the solution (I) to give solution (II); (c) acidifying the solution (II) with inorganic acid for precipitation to give solution (III); and (d) filtering the solution (III) to give Teriflunomide.

### BACKGROUND OF THE INVENTION

Field of the Invention

The present invention relates to a method for preparing Teriflunomide of formula (I).

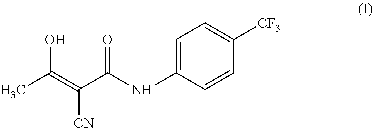 [SMILES: C/C(O)=C(\C#N)C(=O)Nc1ccc(C(F)(F)F)cc1]

Description of Prior Art

The chemical name of Teriflunomide is 2-cyano-3-hydroxy-N[4-(trifluoromethyl)phenyl]-2-butenamide and formula is C₁₂H₉F₃N₂O₂ and molecular weight is 270.207.

Teriflunomide is used as Immunosupressant. It acts as tyrosine kinase inhibitor. It is used in treatment of rheumatoid arthritis, autoimmune disease and multiple sclerosis.

Teriflunomide was first disclosed and claimed in U.S. Pat. No. 5,679,709 but this application does not mention the process of preparation.

U.S. Pat. No. 5,494,911 discloses a process for preparation of Teriflunomide in Example-4 as shown in given below scheme-I

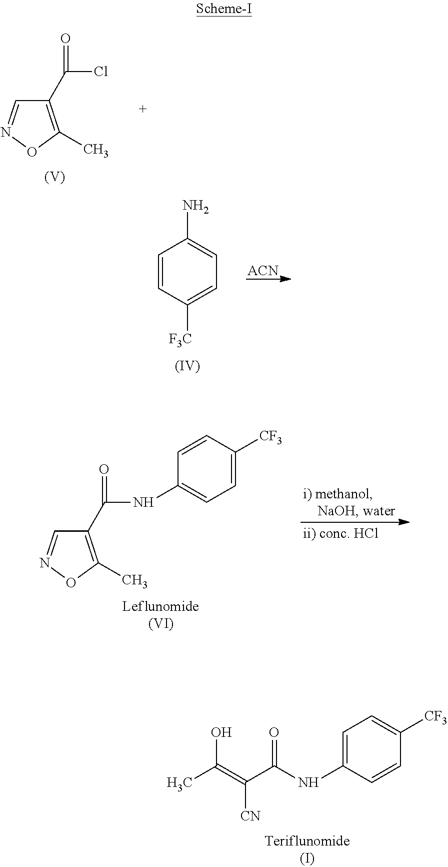

The process involves reacting 5-methylisoxazole-4-carbonyl chloride (V) with 4-trifluoromethylaniline (IV) in acetonitrile to give Leflunomide (VI). The subsequent hydrolysis with aqueous sodium hydroxide solution in methanol gives Teriflunomide (I).

However, the sodium hydroxide in the reaction solution can also act as nucleophile which would further attack the product Teriflunomide to cause the generation of by-product, 4-trifluoromethylaniline, and results in lower yield and poor quality.

4-Trifluoromethylaniline as a genotoxic concerned impurity, should be controlled at a very low level in the final API. Therefore, there is a need to develop a method to overcome the disadvantages of prior art.

### SUMMARY OF THE INVENTION

The present invention provides a method for preparation of Teriflunomide which is devoid of the above mentioned disadvantages and feasible at commercial scale.

The present invention provides a method for preparation of Teriflunomide of formula (I), comprising steps of: (a) adding Leflunomide of formula (II) to an alcoholic solvent to give solution (I); (b) adding an aqueous sodium hydroxide solution slowly into the solution (I) to give solution (II); (c) acidifying the solution (II) with inorganic acid for precipitation of Teriflunomide of formula (I) to give solution (III); and (d) filtering the solution (III) to give Teriflunomide of formula (I).

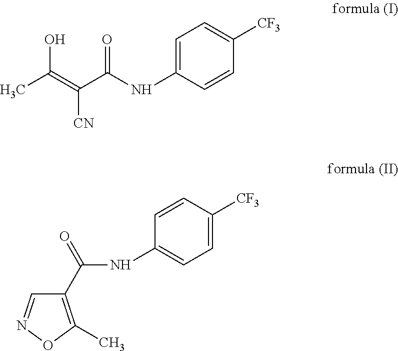 [SMILES: C/C(O)=C(\C#N)C(=O)Nc1ccc(C(F)(F)F)cc1 ; Cc1oncc1C(=O)Nc1ccc(C(F)(F)F)cc1]

### BRIEF DESCRIPTION OF THE DRAWINGS

FIG. 1 shows the synthetic scheme for Teriflunomide from Leflunomide.

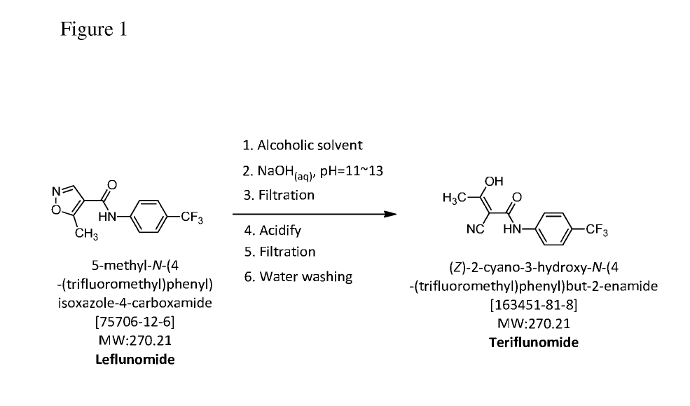

FIG. 2 illustrates HPLC chromatogram of solution (II) to demonstrate that 4-trifluoromethylaniline was not detected at this stage.

FIG. 3 illustrates HPLC chromatogram of the final desired Teriflunomide to demonstrate that 4-trifluoromethylaniline was not detected at this stage.

### DETAILED DESCRIPTION OF THE INVENTION

The invention provides a method for preparation of Teriflunomide of formula (I), comprising steps of:

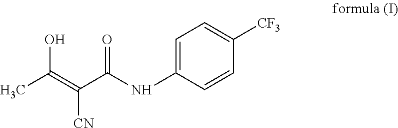 [SMILES: C/C(O)=C(\C#N)C(=O)Nc1ccc(C(F)(F)F)cc1]

(a) adding Leflunomide of formula (II) to an alcoholic solvent to give solution (I);

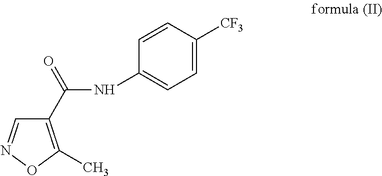 [SMILES: Cc1oncc1C(=O)Nc1ccc(C(F)(F)F)cc1]

(b) adding an aqueous sodium hydroxide solution slowly into the solution (I) to give solution (II),
(c) acidifying the solution (II) with inorganic acid for precipitation to give solution (III); and
(d) filtering the solution (III) to give Teriflunomide of formula (I).

The solution (I) is added slowly with the aqueous sodium hydroxide solution until pH 11 to 13.

In a preferred embodiment, the alcoholic solvent is isopropyl alcohol.

In a preferred embodiment, the inorganic acid is concentrated hydrochloric acid.

In a preferred embodiment, the solution (II) is filtered before acidification.

In a preferred embodiment, the solution (III) is filtered, washed with water, and dried to give Teriflunomide of formula (I).

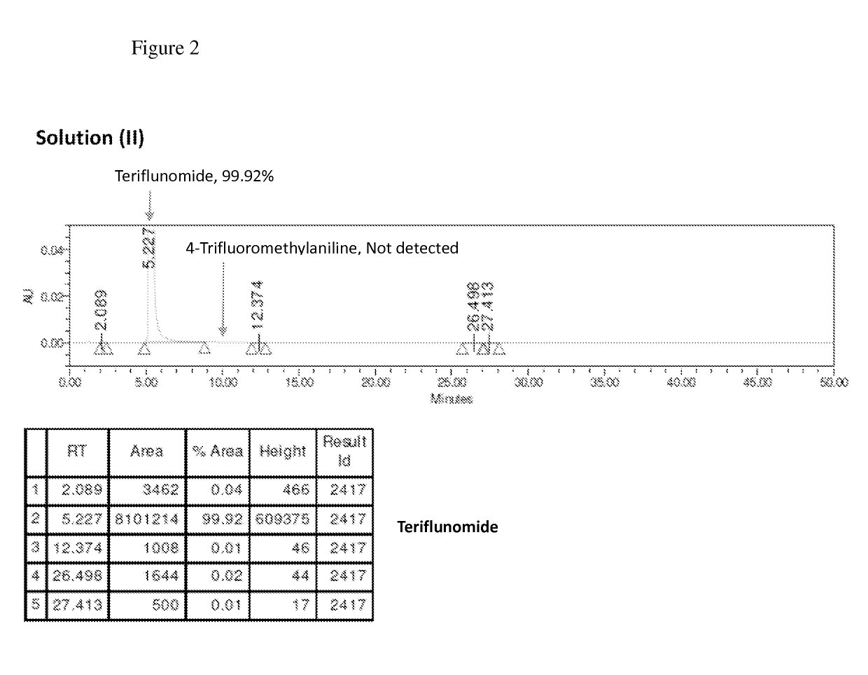

The quality of Teriflunomide of formula (I) obtained in solution (II) from step (b) of the method of the invention has a HPLC purity over 99.8%, and the potential by-product, 4-trifluoromethylaniline, is always not detected (see FIG. 2).

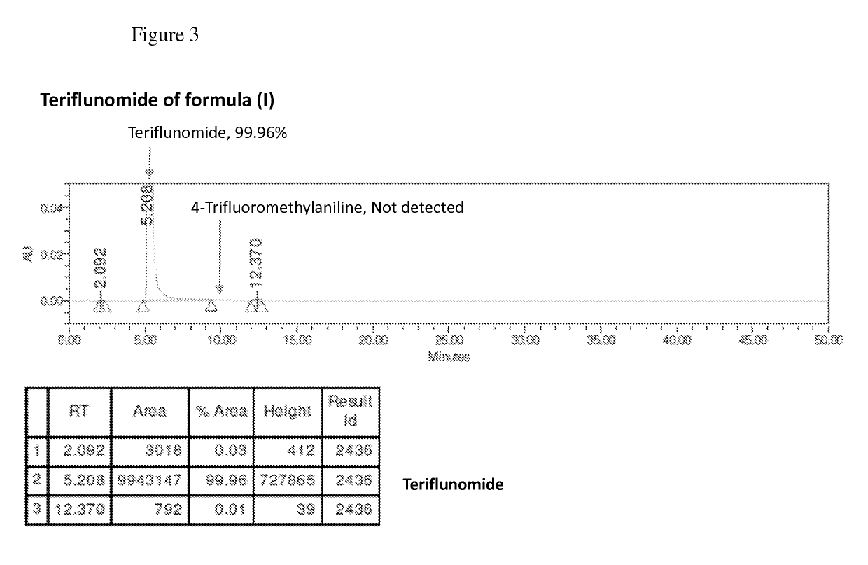

The final quality of Teriflunomide of formula (I) obtained from the method of the invention has a HPLC purity over 99.8%, and the potential by-product, 4-trifluoromethylaniline, is always not detected (see FIG. 3).

No increasing level for content of 4-trifluoromethylaniline is found throughout the whole method.

### EXAMPLES

5-methylisoxazole-4-carboxylic acid (4-trifluoromethyl) anilide (Leflunomide) was added into isopropyl alcohol solution (isopropyl alcohol/water=5/5 (w/w)). To the stirred mixture was added slowly an aqueous sodium hydroxide solution (1.05eq, 45%) at 30±5° C. for 20 to 30 min until the stirred solution reached a pH of 12±1, and then filtered. The solution was acidified with concentrated hydrochloric acid (1.05eq, 32%) at 30±5° C. for 20 to 30 min. The precipitated crystal magma was filtered, washed with water and dried under vacuum to give N-(4-trifluoromethylphenyl)-2-cyano-3-hydroxycrotonamide (Teriflunomide), with HPLC purity over 99.8%. The yield from Leflunomide is about 95%.

This invention will be better understood by reference to the Experimental Details which follow, but those skilled in the art will readily appreciate that the specific experiments detailed are only illustrative of the invention as described more fully in the claims which follow thereafter.

What is claimed is:

1. A method for preparation of Teriflunomide of formula (I), comprising steps of: 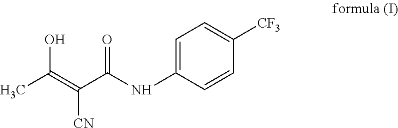 [SMILES: C/C(O)=C(\C#N)C(=O)Nc1ccc(C(F)(F)F)cc1]
(a) adding Leflunomide of formula (II) to an alcoholic solvent to give solution (I); 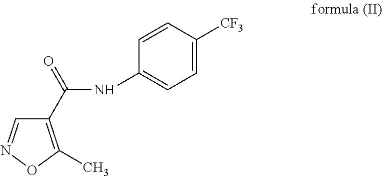 [SMILES: Cc1oncc1C(=O)Nc1ccc(C(F)(F)F)cc1]
(b) adding an aqueous sodium hydroxide solution slowly into the solution (I) to give solution (II);
(c) acidifying the solution (II) with inorganic acid for precipitation to give solution (III); and
(d) filtering the solution (III) to give Teriflunomide of formula (I).

2. The method of claim 1, wherein the solution (I) is added slowly with the aqueous sodium hydroxide solution until pH 11 to 13.

3. The method of claim 1, wherein the alcoholic solvent is isopropyl alcohol.

4. The method of claim 1, wherein the inorganic acid is concentrated hydrochloric acid.

5. The method of claim 1, wherein the solution (III) is filtered, washed with water, and dried to give Teriflunomide of formula (I).

6. The method of claim 1, which no increasing level for content of 4-trifluoromethylaniline is found throughout whole method.
